(19) United States
(12) Patent Application Publication
(10) Pub. No.: US 2025/0352482 A1
(43) Pub. Date: Nov. 20, 2025

(54) AMORPHOUS DOSAGE FORM CONTAINING EBSELEN

(71) Applicant: SOUND PHARMACEUTICALS INC., Seattle, WA (US)

(72) Inventors: Jonathan KIL, Seattle, WA (US); G. Michael WALL, Fort Worth, TX (US)

(21) Appl. No.: 18/872,705

(22) PCT Filed: Jun. 6, 2023
(86) PCT No.: PCT/US2023/068011

§ 371 Date: Dec. 6, 2024

Related U.S. Application Data
(60) Provisional application No. 63/349,474, filed on Jun. 6, 2022.

(51) Int. Cl. A61K 9/48; A61K 9/14; A61K 9/16; A61K 31/41

(52) U.S. Cl. CPC A61K 9/4866; A61K 9/146; A61K 9/1623; A61K 9/1635; A61K 9/1652; A61K 9/4858; A61K 31/41

(57) ABSTRACT

The present disclosure provides an amorphous solid dispersion (ASD) comprising an amorphous form of ebselen. Also provided are pharmaceutical compositions and pharmaceutical dosage forms including the subject amorphous solid dispersion. Also provided are methods of delivering the subject ebselen pharmaceutical dosage forms to a subject to achieve an enhanced maximum blood plasma concentration (Cₘₐₓ) for ebselen with respect to a control ebselen formulation, and an area under the curve (AUC) for both ebselen and an ebselen metabolite that is greater than that achieved with a control ebselen formulation.

### 1. INTRODUCTION

Ebselen (2-Phenyl-1,2-benzoselenazol-3 (2H)-one) is an anti-inflammatory compound with glutathione peroxidase (GPx1) activity of the following structure:

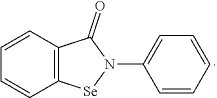 [SMILES: O=c1c2ccccc2[se]n1-c1ccccc1]

GPx1 is the dominant catalytic antioxidant enzyme in the mammalian inner ear, and its activity is decreased after noise or ototoxic insult. Several preclinical and clinical studies have demonstrated the safety and efficacy of SPI-1005 (ebselen) at preventing and treating different forms of sensorineural hearing loss. SPI-1005 capsules, containing 200 mg ebselen, have demonstrated the potential for a safe, well tolerated, oral treatment for Meniere's Disease, a disease for which there are no FDA-approved treatments. Ebselen treatment has been shown to prevent or reverse the pathologic changes in the cochlea following noise- or ototoxin-induced injury, resulting in improved physiology measured by several types of auditory stimuli. Ebselen treatment has been shown to have use in the treatment and/or prevention of chemotherapy induced ototoxicity, and treatment and/or prevention of aminoglycoside-induced ototoxicity in patients with cystic fibrosis. Ebselen treatment has further been shown to have use in treating acute lung infections and related conditions or diseases, such as coronavirus-mediated lung injuries.

Improved oral dosage forms including ebselen are of interest.

### 2. SUMMARY

The present disclosure provides an amorphous solid dispersion (ASD) comprising an amorphous form of ebselen. Also provided are pharmaceutical compositions and pharmaceutical dosage forms including the subject amorphous solid dispersion. Also provided are methods of delivering the subject ebselen pharmaceutical dosage forms to a subject to achieve an enhanced maximum blood plasma concentration (Cₘₐₓ) for ebselen with respect to a control ebselen formulation, and an area under the curve (AUC) for both ebselen and an ebselen metabolite that is greater than that achieved with a control ebselen formulation.

A first aspect of this disclosure includes an amorphous solid dispersion (ASD) comprising an amorphous form of ebselen and a carrier polymer.

A second aspect of this disclosure includes a pharmaceutical composition including the subject amorphous solid dispersion (ASD) and one or more pharmaceutically acceptable excipients.

A third aspect of this disclosure includes a pharmaceutical dosage form including the subject amorphous solid dispersion (ASD), or the subject pharmaceutical composition (e.g., as described herein).

A fourth aspect of this disclosure includes a method of delivering a therapeutically effective amount of ebselen to a subject in need thereof, comprising orally administering to a subject in need thereof a subject pharmaceutical dosage form (e.g., as described herein), to achieve: an enhanced maximum blood plasma concentration (Cₘₐₓ) for ebselen that is at least 2-fold greater than that achieved with a control ebselen formulation; and/or an area under the curve (AUC) for ebselen that is at least 2-fold greater than that achieved with a control ebselen formulation; and/or an area under the curve (AUC) for an ebselen metabolite (e.g., ebselen glucuronide) that is at least 2-fold greater than achieved with a control ebselen formulation.

### 3. BRIEF DESCRIPTION OF THE DRAWINGS

These and other features, aspects, and advantages of the present disclosure will become better understood with regard to the following description, and accompanying drawings where:

FIG. 1 shows plasma concentrations of ebselen after oral (PO) administration of control Formulation 1 (referred to as SPI-1005) or Formulation 2, ebselen amorphous spray-dried dispersion hot melt extrusion formulation (referred to as ASD/HME).

FIG. 2 shows plasma concentrations of ebselen glucuronide after oral (PO) administration of control Formulation 1 (referred to as SPI-1005) or Formulation 2, ebselen amorphous spray-dried dispersion hot melt extrusion formulation (referred to as ASD/HME).

### 4. DETAILED DESCRIPTION

As summarized above, the present disclosure provides an amorphous solid dispersion (ASD) comprising an amorphous form of ebselen. Also provided are pharmaceutical compositions and pharmaceutical dosage forms including the subject amorphous solid dispersion. Also provided are methods of delivering the subject ebselen pharmaceutical dosage forms to a subject to achieve an enhanced maximum blood plasma concentration (Cₘₐₓ) for ebselen with respect to a control ebselen formulation, and/or an area under the curve (AUC) for both ebselen and an ebselen metabolite that is greater than that achieved with a control ebselen formulation.

The present application further provides pharmaceutical compositions and dosage forms of ebselen having enhanced bioavailability in comparison to a control ebselen formulation (e.g., as described herein).

The amorphous solid dispersion (ASD) of this disclosure, and pharmaceutical compositions comprising the ASD are described in greater detail below. Also described are pharmaceutical dosage forms comprising the subject ASD or pharmaceutical compositions, and methods for delivering the same to a subject.

### 4.1. Amorphous Solid Dispersion (ASD)

As summarized above, this disclosure provides an amorphous solid dispersion (ASD) comprising an amorphous form of ebselen.

### 4.1.1. Ebselen

The subject pharmaceutical compositions include ebselen (2-Phenyl-1,2-benzoselenazol-3 (2H)-one), an anti-inflammatory compound with glutathione peroxidase (GPx1) activity of the following structure:

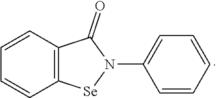 [SMILES: O=c1c2ccccc2[se]n1-c1ccccc1]

Ebselen (International Non-proprietary Name (INN)) is also known as 2-phenyl-1,2-benzisoselenazol-3 (2H)-one (chemical name), 60940-34-3 (Chemical Abstracts Service (CAS) Number), SPI-1005, C05110703, C05110703-B, N0285 (Sound Pharmaceuticals, Inc., code numbers), PZ 51 (A.Natterman & Cie GmbH (Cologne, F.R.G.) Code Number), DR3305 (Watanabe, 1997), ebselene, ebselenum, ebseleno and harmokisane.

### 4.1.2. Amorphous Solid Dispersion Formation

The term “solid dispersion” refers to a system in a solid-state comprising at least two components, wherein one component is dispersed throughout the other component or components.

The solid dispersions of ebselen may be formed by any conventional technique, e.g., spray drying, co-grinding, hot melt extrusion, freeze drying, rotary evaporation, solvent evaporation, co-precipitation, lyophilization, or any suitable solvent removal process. In an embodiment, solid dispersions of ebselen of the present application comprises amorphous forms of ebselen, and a pharmaceutically acceptable carrier.

The ebselen starting material used in the process for preparation of the solid dispersion may be crystalline or amorphous form. Alternatively, it may be obtained in situ from a previous processing step.

The ebselen in the solid dispersion obtained is present in an amorphous form.

A solid that is in the “amorphous” solid state form means that it is in a non-crystalline state. Amorphous solids generally possess crystal-like short-range molecular arrangement, but no long-range order of molecular packing as are found in crystalline solids. The solid-state form of a solid, such as the drug substance in the amorphous dispersion, may be determined by Polarized Light Microscopy, X-Ray Powder Diffraction (XPRD), Differential Scanning calorimetry (DSC), or other standard techniques known to those of skill in the art. In some embodiments, the amorphous solid contains ebselen in a substantially amorphous solid-state form, e.g., at least about 80% of ebselen in the dispersion is in an amorphous form, such as at least about 90% of ebselen in the dispersion is in an amorphous form, or at least about 95% of ebselen in the dispersion is in amorphous form.

In some embodiments, at least about 90% (e.g., at least 95%, 96%, 97%, 98%, 99%, 99.5%, or even 99.9%, such as from 90% to 99.9%, from 90% to 99.5%, from 90% to 99%, from 90% to 98%, from 90% to 97%, from 90% to 96%, from 90% to 95%, from 95% to 99.9%, from 95% to 99.5%, from 95% to 99%, from 95% to 98%, from 95% to 97%, and from 95% to 96%) of the ebselen is in amorphous form.

The solid dispersion can be in a single phase such as substitutional or interstitial amorphous solutions; or it can be a two-phase system such as eutectics, amorphous drug and amorphous carrier dispersions. Solid solutions are a resultant single phase upon dispersion of two compounds in each other, at their molecular level.

The inventors of the present application have found that a composition comprising amorphous solid dispersion of ebselen comprising at least one pharmaceutically acceptable carrier, can increase the maximum blood plasma concentration (Cₘₐₓ) and area under the curve (AUC) of ebselen by at least 2-fold compared to a control ebselen formulation.

In some embodiments, the amorphous solid dispersion (ASD) of ebselen includes a carrier polymer.

According to some embodiments of the disclosure, the carrier polymer is selected from one or more of a hydroxypropyl methyl cellulose acetate succinate (HPMC-AS), polyvinyl pyrrolidine and vinyl acetate (PVP/VA) copolymer, hydroxypropyl methylcellulose phthalate (HPMCP), hydroxypropyl methylcellulose (HPMC), polyethylene glycol (PEG), hydroxypropyl cellulose (HPC), carboxymethyl cellulose (CMC), and polyvinyl pyrrolidine (PVP).

In some embodiments, the carrier polymer is selected from polyvinylpyrrolidone polymer (e.g., PVP K30), a copovidone polymer (e.g., PVP VA64 or Kollidon VA64), a hydroxypropyl methyl cellulose polymer (e.g., HPMC E3 or HPMC ASLF), a polyvinyl caprolactam-polyvinyl acetate-polyethylene glycol graft co-polymer (e.g., soluplus), a dimethylaminoethyl methacrylate-copolymer (e.g., Eudragit EPO), a methacrylic acid-methyl methacrylate copolymer (e.g., Eudragit L100), and a polyethylene glycol polymer (e.g., PEG 8000)

According to the embodiments of the invention, the hydroxypropyl methylcellulose acetate succinate (HPMC-AS) comprises various types, such as LF, LG, MF, MG, HF and HG, etc., the first letters L, M and H of the type's names mean the pH level at the beginning of dissolution of HPMC-AS. For example, L refers to low level (e.g., HPMC-AS begins to be dissolved when the pH value is more than 5.5), M refers to middle level (e.g., HPMC-AS begins to be dissolved when the pH value is more than 6.0), H refers to high level (e.g., HPMC-AS begins to be dissolved when the pH value is more than 6.5). The second letters F and G refer to the particle size of HPMC-AS, where F refers to fine powder, and G refers to granular. In some embodiments, the type of HPMC-AS is LF; in some embodiments, the type of HPMC-AS is MF; in some embodiments, the type of HPMC-AS is HG.

More generally, any convenient carrier polymer can find use in subject ASD formulations. In some embodiments, the carrier polymers include but are not limited to, cellulose acetate phthalate, cellulose acetate trimellitate, cellulose acetate succinate, methyl cellulose phthalate, ethylhydroxymethylcellulose phthalate, hydroxypropylmethylcellulose phthalate (HPMCP), hydroxypropylmethyl cellulose acetate succinate (HPMC-AS), hydroxypropylmethyl cellulose acetate maleate, hydroxypropylmethylcellulose trimellitate, carboxymethylethyl cellulose, polyvinyl butyrate phthalate, polyvinyl acetate phthalate, a methacrylic acid/ethyl acrylate copolymer and a methacrylic acid/methyl methacrylate copolymer. In some embodiments, the polymer is selected from HPMCP, HPMC-AS, hydroxypropylmethyl cellulose acetate maleate and hydroxypropylmethylcellulose trimellitate.

In some embodiments, the carrier polymer is selected from the group consisting of hydroxypropyl methylcellulose, hydroxypropyl cellulose, hydroxyethyl cellulose, polyvinylpyrrolidone (povidone), poly(vinylpyrrolidone/vinylacetate) (copovidone), polyvinylcaprolactam/polyvinylacetate/polyethylene glycol graft copolymer, polyethylene glycol/polyvinyl alcohol graft copolymer, polyethylene oxide, polypropylene oxide, copolymers of ethylene oxide and propylene oxide, polyvinyl alcohol, partially saponified polyvinylalcohol, macrogolglycerol hydroxystearate, polyethylene glycol, and maltodextrins. In some embodiments, the carrier polymer is a copovidone polymer.

In some embodiments, the amorphous solid dispersion of the amorphous ebselen, and polymer carrier have a weight ratio of the ebselen to the polymer carrier of from about 1:6 to about 1:1 (e.g., from 1:6 to 1:2, from 1:6 to 1:2.5, from 1:6 to 1:3, from 1:6 to 1:3.5, from 1:6 to 1:4, from 1:6 to 1:4.5, from 1:6 to 1:5, from 1:5 to 1:2, from 1:5 to 1:2.5, from 1:5 to 1:3, from 1:5 to 1:3.5, from 1:5 to 1:4, from 1:5 to 1:4.5, from 1:5 to 1:1.5, from 1:4 to 1:1.5, from 1:4 to 1:2, from 1:4 to 1:2.5, from 1:4 to 1:3, from 1:4 to 1:3.5, from 1:3 to 1:1.5, from 1:3 to 1:2, from 1:3 to 1:2.5, and from 1:2 to 1:1.5).

In certain embodiments, ebselen and carrier polymer are present in a ratio of from 1:1 to 1:6 (w/w), 1:1 to 1:4 (w/w), such as in the ratio of 1:4 (w/w).

Solid dispersions of the present invention optionally may include one or more solubilizers, i.e., additives which increase solubility of the pharmaceutical active ingredient in the solid dispersion or additives which act as pore-forming agents in the solid dispersion. Suitable solubilizers for use in compositions of the present disclosure include mannitol, transcutol, polyvinylalcohol, hydroxypropyl methylcellulose, hydroxypropyl cellulose, methyl cellulose, polyvinylpyrrolidone, glycofurol and transcutol. The concentration of solubilizer ranges from about 0.5% to about 30% w/w of carrier concentration.

The amorphous solid dispersions of the present disclosure optionally may include one or more surfactants. Surfactants are compounds which are capable of improving the wetting of the pharmaceutical active ingredient and/or enhancing the dissolution. The surfactants can be selected from hydrophilic surfactants or lipophilic surfactants or mixtures thereof. The surfactants can be anionic, nonionic, cationic, and zwitterionic surfactants. Surfactants according to the present disclosure include, but not limited to, polyoxyethylene alkylaryl ethers such as polyoxyethylene lauryl ether, polyoxyethylene cetyl ether, polyoxyethylene stearyl ether; polyethylene glycol fatty acid esters such as PEG monolaurate, PEG dilaurate, PEG distearate, PEG dioleate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, sodium lauryl sulfate, sodium dioctyl sulfosuccinate (DOSS), lecithin, stearylic alcohol, cetostearylic alcohol, cholesterol, polyoxyethylene ricin oil, polyoxyethylene fatty acid glycerides, cremophor RH 40, and the like or combinations thereof. The concentration of surfactant ranges from about 0.1% to about 10% w/w of carrier concentration.

In some embodiments herein, the percentage loading of ebselen in the solid dispersion is from about 1% to about 90% (w/w) (e.g., from 1% to 19%, from 10% to 19%, from 10% to 20%, from 10% to 30%, from 10% to 40%, from 10% to 50%, from 10% to 60%, from 10% to 70%, from 10% to 80%, from 10% to 90%, from 20% to 30%, from 20% to 40%, from 20% to 50%, from 20% to 60%, from 20% to 70%, from 20% to 80%, from 20% to 90%, from 21% to 30%, from 21% to 34%, from 21% to 40%, from 21% to 50%, from 21% to 60%, from 21% to 70%, from 21% to 80%, from 21% to 90%, from 30% to 40%, from 30% to 50%, from 30% to 60%, from 30% to 70%, from 30% to 80%, from 30% to 90%, from 36% to 40%, from 36% to 49%, from 36% to 60%, from 36% to 70%, from 36% to 80%, from 36% to 90%, from 40% to 50%, from 40% to 60%, from 40% to 70%, from 40% to 80%, from 40% to 90%, from 50% to 60%, from 50% to 70%, from 50% to 80%, from 50% to 90%, 51% to 60%, from 51% to 70%, from 51% to 80%, from 51% to 90%, from 60% to 70%, from 60% to 80%, from 60% to 90%, from 70% to 80%, and from 70% to 90%). In some embodiments, the percentage loading of ebselen is from about 10% to about 60% (w/w) (e.g., from 10% to 20%, from 10% to 30%, from 10% to 40%, from 10% to 50%, from 10% to 60%, from 20% to 30%, from 20% to 40%, from 20% to 50%, from 20% to 60%, from 30% to 40%, from 30% to 50%, from 30% to 60%, from 40% to 50%, and from 40% to 60%).

In some embodiments, the percentage loading of ebselen in the solid dispersion is from 10-30% w/w. In some embodiments, the percentage loading of ebselen in the solid dispersion is from 15-25% w/w ebselen, such as 15%, 16%, 17%, 18%, 19%, 20%, 21%, 22%, 23%, 24%, or 25% w/w ebselen. In some embodiments, the percentage loading of ebselen in the solid dispersion is 20% w/w.

Solid dispersions of the present disclosure can be obtained by one or more of methods selected from spray-drying, solvent evaporation, co-precipitation, hot melt extrusion, co-grinding and lyophilization. The solid dispersions obtained by the present disclosure are present either in amorphous form.

In an embodiment, amorphous solid dispersions of ebselen are obtained by hot melt extrusion. The term hot-melt extrusion or hot-melt extruded is used herein to describe a process whereby a composition is heated and/or compressed to a molten (or softened) state and subsequently forced through an orifice in a die where the extruded product is formed into its final shape in which it solidifies upon cooling. The blend is conveyed through one or more heating zones typically by a screw mechanism. The screw or screws are rotated by a variable speed motor inside a cylindrical barrel where only a small gap exists between the outside diameter of the screw and the inside diameter of the barrel. In this conformation, high shear is created at the barrel wall and between the screw fights by which the various components of the powder blend are well mixed and disaggregated. The die can be a dual manifold, multi-manifold or feed-block style die.

In an embodiment, amorphous solid dispersions of ebselen are obtained by a spray drying process. Spray dried dispersions are obtained by dissolving drug and the carrier polymer in an organic solvent and then spray-drying the solution. The formulation and process conditions are chosen so that the solvent quickly evaporates from the droplets, allowing insufficient time for phase separation or crystallization.

In an embodiment, ebselen and at least one carrier polymer is mixed with one or more of organic solvents. Suitable solvents for mixing are selected from methanol, ethanol, isopropanol (IPA), ethyl acetate, dichloromethane (DCM), ethylene chloride, chloroform, acetonitrile, acetone, tetrahydrofuran (THF) and mixtures thereof.

The resultant amorphous solid dispersions of ebselen can be blended with one or more excipients, as described herein, and then granulated and/or compacted to produce a final blend for encapsulating or tableting.

In particular embodiments, the amorphous solid dispersion of ebselen may be combined with one or more excipient(s) may be included to form granules, e.g., such as a binding agent, a filler, a disintegrating agent, a wetting agent, a glidant, and a lubricant.

### 4.2. Pharmaceutical Compositions

As summarized above, this disclosure provides pharmaceutical compositions including the subject amorphous solid dispersion (ASD) comprising an amorphous form of ebselen.

The subject amorphous solid dispersion may be used for filling any one of the unit dosage forms described herein (e.g., a capsule) or for tableting. The amorphous solid dispersion can optionally be further processed before filling or tableting. Exemplary further processing includes spheronizing, pelletizing, milling, injection molding, sieving, and/or calendering the solid dispersion.

Amorphous solid dispersions of ebselen of the present disclosure can be optionally subjected to a particle size reduction procedure before or after the completion of drying of the product to produce desired particle sizes and distributions. Milling or micronization can be performed to achieve the desired particle sizes or distributions. Equipment that may be used for particle size reduction include, without limitation thereto, ball mills, roller mills, hammer mills, and jet mills.

In one embodiment, there is provided amorphous solid dispersions of ebselen comprising amorphous form of ebselen wherein 90% of the particles are less than about 500 microns or less than about 200 microns or less than about 100 microns or less than about 50 microns or less than about 40 microns or less than about 30 microns or less than about 20 microns or less than about 10 microns or any other suitable particle sizes.

The amorphous solid dispersion of ebselen may be combined with pharmaceutically acceptable excipients to make other pharmaceutical compositions, or a finished dosage form (e.g., as described herein). The one or more additional pharmaceutically acceptable excipients are selected from diluents, binders, disintegrants, lubricants, glidants, surfactants, solubilizers, stabilizing agents, antioxidants, colors, flavors, preservatives, and combinations thereof.

Other pharmaceutically acceptable excipients may include, but are not limited to, diluents, binders, disintegrating agents, surfactants, plasticizers, lubricants, glidants, chelating agents, coating agents and the like or mixtures thereof as extra-granular agents.

### 4.2.1. Excipients

The pharmaceutical compositions provided in accordance with the present disclosure can be administered orally. In certain embodiments, the disclosure provides pharmaceutical compositions that include a subject amorphous solid dispersion as described herein, and one or more pharmaceutically acceptable excipients or carriers including but not limited to, inert solid diluents and fillers, diluents, including sterile aqueous solution and various organic solvents, permeation enhancers, solubilizers, surfactants, disintegrants, lubricants, binders, glidants, adjuvants, and combinations thereof. Such compositions are prepared in a manner well known in the pharmaceutical art (see, e.g., Remington: The Science and Practice of Pharmacy (Remington: The Science and Practice of Pharmacy, 23rd Edition, ISBN-13:978-0128200070); and Modern Pharmaceutics, Marcel Dekker, Inc., 4th Ed. (G. S. Banker & C. T. Rhodes, Eds.).

The pharmaceutical compositions may be administered by oral administration. Administration may be via capsule, tablet, or the like. In one embodiment, the ebselen formulation is in the form of a tablet. In a further embodiment, the tablet is a compressed tablet. In making the pharmaceutical compositions that include the solid described herein, the active ingredient is usually diluted by an excipient and/or enclosed within such a carrier that can be in the form of a capsule, tablet, sachet, or other container. When the excipient serves as a diluent, it can be in the form of a solid, semi-solid or liquid material (as above), which acts as a vehicle, carrier or medium for the active ingredient.

The pharmaceutical composition may be formulated for immediate release or sustained release. A “sustained release formulation” is a formulation which is designed to slowly release a therapeutic agent in the body over an extended period of time, whereas an “immediate release formulation” is a formulation which is designed to quickly release a therapeutic agent in the body over a shortened period of time. In some cases, the immediate release formulation may be coated such that the therapeutic agent is only released once it reached the desired target in the body (e.g., the stomach). In a specific embodiment, the pharmaceutical composition is formulated for immediate release.

The pharmaceutical composition may further comprise pharmaceutical excipients such as fillers or diluents, binders, glidants, disintegrants, lubricants, solubilizers, and combinations thereof. Some examples of suitable excipients are described herein. When the pharmaceutical composition is formulated into a tablet, the tablet may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, the pharmaceutical composition comprises a filler, such as a carbohydrate or a protein filler. In some embodiments, the filler is selected from the group consisting of dicalcium phosphate, cellulose, microcrystalline cellulose, hydroxypropylmethyl cellulose, sodium carboxymethylcellulose, compressible sugars, dibasic calcium phosphate dehydrate, lactose, lactose monohydrate, sucrose, mannitol, sorbital, starch from corn, wheat, rice, potato or other plants, tribasic calcium phosphate, a gum (e.g., Arabic or tragacanth), proteins (e.g., gelatin or collagen) and combinations thereof.

In further embodiments, the pharmaceutical composition comprises one or more fillers in an amount from about 10 to about 65% w/w, or from about 10 to about 60% w/w, or from about 10% to about 55% w/w, or from about 10 to about 50% w/w, or from about 20 to about 50% w/w, or from about 25 to about 50% w/w, or from about 25 to about 45% w/w, or from about 25 to 40% w/w, or from about 30 to about 40%, or about 34 to about 40% w/w. In specific embodiments, one or more fillers is present in an amount of about 25%, or about 22%, or about 24%, or about 26%, or about 28%, or about 30%, or about 32%, or about 34%, or about 36%, or about 38%, or about 40 w/w. In a further specific embodiment, the filler is microcrystalline cellulose present in an amount of about 35% w/w. In a further specific embodiment, the filler is microcrystalline cellulose present in an amount of about 38% w/w.

In further embodiments, the pharmaceutical composition comprises one or more fillers in an amount from 10 to 65% w/w, or from 10 to 60% w/w, or from 10% to 55% w/w, or from 10 to 50% w/w, or from 20 to 50% w/w, or from 25 to 50% w/w, or from 25 to 45% w/w, or from 25 to 40% w/w, or from 30 to 40%, or 34 to 40% w/w. In specific embodiments, the one or more fillers is present in an amount of 25%, or 22%, or 24%, or 26%, or 28%, or 30%, or 32%, or 34%, or 36%, or 38%, or 40 w/w. In a further specific embodiment, the filler is microcrystalline cellulose present in an amount of 35% w/w. In a further specific embodiment, the filler is microcrystalline cellulose present in an amount of 38% w/w.

In some embodiments, the pharmaceutical composition comprises a disintegrant or solubilizing agent selected from cross-linked polyvinyl pyrrolidone, agar, alginic acid or a salt thereof (e.g., sodium alginate), croscarmellose sodium, crospovidone, modified corn starch, pregelatinized starch, sodium starch glycolate, and combinations thereof.

In certain embodiments, the pharmaceutical composition comprises one or more disintegrants in an amount from about 1 to about 10% w/w, or from about 1 to about 9% w/w, or from about 1 to about 8% w/w, or from about 2 to about 8% w/w, or from about 4 to about 8% w/w. In specific embodiments, one or more disintegrants is present in an amount of about 1%, or about 2%, or about 3%, or about 4%, or about 5%, or about 6%, or about 7%, or about 8%, or about 9%, or about 10% w/w. In a further specific embodiment, the disintegrant is croscarmellose sodium present in an amount of about 4% w/w. In a further specific embodiment, the disintegrant is croscarmellose sodium present in an amount of about 2% w/w. In a further specific embodiment, the disintegrant is croscarmellose sodium present in an amount of about 8% w/w.

In certain embodiments, the pharmaceutical composition comprises one or more disintegrants in an amount from about 1 to 10% w/w, or from 1 to 9% w/w, or from 1 to 8% w/w, or from 2 to 8% w/w, or from 4 to 8% w/w. In specific embodiments, one or more disintegrants is present in an amount of 1%, or 2%, or 3%, or 4%, or 5%, or 6%, or 7%, or 8%, or 9%, or 10% w/w. In a further specific embodiment, the disintegrant is croscarmellose sodium present in an of 4% w/w. In a further specific embodiment, the disintegrant is croscarmellose sodium present in an of 2% w/w. In a further specific embodiment, the disintegrant is croscarmellose sodium present in an of 8% w/w.

In some embodiments, the pharmaceutical composition comprises a lubricant selected from the group consisting of calcium stearate, magnesium stearate, polyethylene glycol, sodium stearyl fumarate, stearic acid, and combinations thereof.

In further embodiments, the pharmaceutical composition comprises one or more lubricants in an amount from about 0.1 to about 1% w/w, or from about 0.1 to about 0.7% w/w, or from about 0.25 to about 1% w/w, or from about 0.25 to about 0.7% w/w, or from about 0.25 to about 0.6% w/w, or from about 0.25 to about 0.5% w/w, or from about 0.3 to about 0.5% w/w. In specific embodiments, the lubricant is magnesium stearate present in an amount of about 0.1%, or about 0.2, or about 0.3%, or about 0.4%, or about 0.5% w/w. In a further specific embodiment, the lubricant is magnesium stearate present in an amount of about 0.5% w/w.

In further embodiments, the pharmaceutical composition comprises one or more lubricants in an amount from 0.1 to 1% w/w, or from 0.1 to 0.7% w/w, or from 0.25 to 1% w/w, or from 0.25 to 0.7% w/w, or from 0.25 to 0.6% w/w, or from 0.25 to 0.5% w/w, or from 0.3 to 0.5% w/w. In specific embodiments, the lubricant is magnesium stearate present in an amount of 0.1%, or 0.2, or 0.3%, or 0.4%, or 0.5% w/w. In a further specific embodiment, the lubricant is magnesium stearate present in an amount of 0.5% w/w.

In some embodiments, the pharmaceutical composition comprises a glidant selected from the group consisting of colloidal silicon dioxide, talc, and combinations thereof.

In further embodiments, the pharmaceutical composition comprises one or more glidants in an amount from about 0.1 to about 0.5% w/w, or from about 0.2 to about 0.5% w/w, or from about 0.3 to about 0.5% w/w. In specific embodiments, one or more glidants is present in an amount of about 0.1% w/w, 0.2% w/w, 0.3% w/w, 0.4% w/w, 0.5% w/w. In a further specific embodiment, the glidant is colloidal silicon dioxide present in an amount of about 0.5% w/w.

In further embodiments, the pharmaceutical composition comprises one or more glidants in an amount from 0.1 to 0.5% w/w, or from 0.2 to 0.5% w/w, or from 0.3 to 0.5% w/w. In specific embodiments, one or more glidants is present in an amount of 0.1% w/w, 0.2% w/w, 0.3% w/w, 0.4% w/w, 0.5% w/w. In a further specific embodiment, the glidant is colloidal silicon dioxide present in an amount of 0.5% w/w.

In some embodiments, the pharmaceutical composition comprises one or more surfactants. Suitable surfactants include both non-ionic and ionic (cationic, anionic and zwitterionic) surfactants suitable for use in pharmaceutical dosage forms. These include polyethoxylated fatty acids and its derivatives, for example, polyethylene glycol 400 distearate, polyethylene glycol-20 dioleate, polyethylene glycol 4-150 mono dilaurate, and polyethylene glycol-20 glyceryl stearate; alcohol-oil transesterification products, for example, polyethylene glycol-6 corn oil; polyglycerized fatty acids, for example, polyglyceryl-6 pentaoleate; propylene glycol fatty acid esters, for example, propylene glycol monocaprylate; mono and diglycerides, for example, glyceryl ricinoleate; sterol and sterol derivatives; sorbitan fatty acid esters and its derivatives, for example, polyethylene glycol-20 sorbitan monooleate and sorbitan monolaurate; polyethylene glycol alkyl ether or phenols, for example, polyethylene glycol-20 cetyl ether and polyethylene glycol-10-100 nonyl phenol; sugar esters, for example, sucrose monopalmitate; polyoxyethylene-polyoxypropylene block copolymers known as “poloxamer;” ionic surfactants, for example, sodium caproate, sodium glycocholate, soy lecithin, sodium stearyl fumarate, propylene glycol alginate, octyl sulfosuccinate disodium, and palmitoyl carnitine; and the like and mixtures thereof. The concentration of surfactant ranges from about 0.5% to about 10% w/w of total composition.

In some embodiments, the pharmaceutical composition may include one or more plasticizers. Suitable plasticizers include polyethylene glycol, propylene glycol, polyethylene oxide, 1,2-butylene glycol, 2,3-butylene glycol, styrene glycol, diethylene glycol, triethylene glycol, tetraethylene glycol and monoisopropyl ether, propylene glycol monoethyl ether, ethylene glycol monoethyl ether, diethylene glycol monoethyl ether, sorbitol lactate, ethyl lactate, butyl lactate, ethyl glycolate, triethyl citrate, acetyl triethyl citrate, tributyl citrate and allyl glycolate. The concentration of plasticizer ranges from about 0.5% to about 10% w/w of total composition.

In some embodiments, the pharmaceutical composition may include a coloring agent. Suitable coloring agents include dyes and pigments such as iron oxide red or yellow, titanium dioxide, talc. The concentration of coloring agent can range from about 0.1% to about 1% w/w of the total composition.

In some embodiments, the pharmaceutical composition may include a chelating agent. Suitable chelating agents include, one or more of, but not limited to ethylenediaminetetraacetic acid (EDTA), disodium EDTA and derivatives thereof, citric acid and derivatives thereof, niacinamide and derivatives thereof, and sodium desoxycholate and the like or mixtures thereof. The concentration of chelating agent can range from about 0.1% to about 1% w/w of total composition.

The pharmaceutical compositions described herein can be formulated with ebselen as the sole pharmaceutically active ingredient in the composition or can be combined with other active ingredients (e.g., as described herein).

In certain embodiments, the pharmaceutical composition is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

Concentrations of the ebselen in a pharmaceutical composition provided herein will depend on, e.g., the physicochemical characteristics of the compound, the dosage schedule, and amount administered as well as other factors known to those of skill in the art. For example, if the composition comprises a salt of ebselen the amount of said salt to be administered and/or to be incorporated into a pharmaceutical composition (i.e., pharmaceutical dosage form) needs to be adjusted to take account of the molecular weight difference between the free base and salt form. For instance, in expressing dose amounts in the label and/or product information of authorized medicinal products comprising a salt form of an active compound that can also be used in free base form, it is customary practice to specify the dose of the free base to which the dose of the salt as used is equivalent.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of ebselen or pharmaceutically acceptable derivatives thereof. The ebselen is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of ebselen sufficient to produce the desired therapeutic effect, in association with the required pharmaceutical carrier, vehicle or filler. Examples of unit-dose forms include ampoules and syringes and individually packaged tablets. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In some embodiments the subject pharmaceutical composition is formulated as tablets, pills, dragees, capsules, liquids, gels, syrups, slurries, suspensions, and the like, suitable for ingestion by a subject. In certain cases, the pharmaceutical composition is formulated as a dragee, and dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the dragee coatings for product identification or to characterize the quantity of active compound (i.e., dosage).

In some embodiments, the subject pharmaceutical composition is formulated for oral use as push-fit capsules made of gelatin, as well as soft, sealed capsules made of gelatin having a coating such as glycerol or sorbitol. Push-fit capsules can contain ebselen mixed excipients as described herein, e.g., fillers or binders such as lactose or starches, lubricants such as talc or magnesium stearate, and optionally stabilizers. In soft capsules, the ebselen may be dissolved or suspended in suitable liquids, such as fatty oils, liquid paraffin, or liquid polyethylene glycol with or without stabilizers.

In certain embodiments, the ebselen described herein are in a liquid pharmaceutical formulation. Liquid pharmaceutically administrable formulations can, for example, be prepared by dissolving, dispersing, or otherwise mixing the active compounds and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (Remington: The Science and Practice of Pharmacy, 23rd Edition, ISBN-13:978-0128200070) Dosage forms or compositions containing ebselen in the ranges disclosed herein with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing the combination described herein is an effective mode of administration. Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a compound provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the pharmaceutical composition is formulated in as a solid dosage form, such as a tablet (e.g., as described herein below).

In some embodiments, the disclosure relates to pharmaceutical compositions comprising amorphous solid dispersions of ebselen for oral administration, which solid dispersion comprises at least one carrier polymer.

In some embodiments, the present disclosure relates to pharmaceuticals compositions as described herein, which are stable, e.g., stable over the shelf life of the drug product. As used herein, the term “stable” is defined as no more than about 5% loss of ebselen under typical commercial storage conditions. In certain embodiments, the formulations of the present invention will have no more than about 3% loss of ebselen, such as, no more than about 2% loss of ebselen, under typical commercial storage conditions. The composition retains at least about 95% of the potency of ebselen after storing the composition at 40° C. and 75% relative humidity for at least three months. In certain aspects, the term “stable” refers to chemical stability, wherein not more than 1.5% w/w of total related substances are formed on storage at accelerated conditions of stability at 40° C. and 75% relative humidity or at 25° C. and 60% relative humidity for a period of at least three months or to the extent necessary for use of the composition.

### 4.2.2. Additional Active Agents

The pharmaceutical compositions disclosed herein may optionally include one or more additional active agents. In some embodiments, at least one of the one or more additional active agents is an otoprotectant agent. In some embodiments, at least one of the one or more additional active agents is N-acetylcysteine or allopurinol.

Some otoprotectant agents that can be included in the subject pharmaceutical compositions include glutathione and glutathione precursors. Examples of otoprotectant agents in this category include but are not limited to, methionine, N-acetyl-DL-methionine, S-adenosylmethionine, cysteine, homocysteine, N-acetylcysteine, glutathione, glutathione ethylester, glutathione diethylester, glutathione triethylester, cysteamine, cystathione, N,N′-diacetyl-L-cystine (DiNAC), 2 (R,S)-D-ribo-(1′,2′,3′,4′-tetrahydroxybutyl)-thiazolidine-4 (R)-carboxylic acid (RibCys), 2-alkylthiazolidine 2 (R,S)-D-ribo-(1′,2′,3′,4′-tetrahydroxybutyl) thiazolidine (RibCyst), and 2-oxo-L-thiazolidine-4-carboxylic acid (OTCA).

Xanthine oxidase inhibitors, for example allopurinol and its tautomers, can also be included in the subject pharmaceutical compositions. Examples of allopurinol derivatives include but are not limited to, 1-methylallopurinol, 2-methylallopurinol, 7-methylallopurinol, 1,5-dimethylallopurinol, 2,5-dimethylallopurinol, 1,7-dimethylallopurinol, 2,7 dimethylallopurinol, 5,7-dimethylallopurinol, 2,5,7-trimethylallopurinol, 1-ethoxycarbonylallopurinol, and 1-ethoxycarbonyl-5-methylallopurinol.

Additional glutathione peroxidase mimics can also be included in the subject pharmaceutical compositions. Examples of additional glutathione peroxidase mimics include but are not limited to, 6A,6B-diseleninic acid-6A′,6B′-selenium bridged-cyclodextrin (6-diSeCD), and 2,2′-diseleno-bis-Beta-cyclodextrin (2-diSeCD).

In some embodiments, the combination of ebselen, and one or more additional active agents according to the present disclosure can allow for a reduction in the dose of ebselen.

A dose reduction can be beneficial for patients which otherwise would potentially suffer from side effects in a therapy using a higher dose of ebselen. As such, the pharmaceutical composition and methods according to the present disclosure can exhibit less side effects to a corresponding monotherapy with ebselen, thereby making the therapy more tolerable and improving an individual's compliance with the treatment.

In certain embodiments, the pharmaceutical composition comprises from 1 to 50% w/w of an additional active agent, such as 1 to 45% w/w, 1 to 40% w/w, 1 to 35% w/w, 1 to 30% w/w, 1 to 25% w/w, 1 to 20% w/w, 1 to 15% w/w, 1 to 10% w/w, or 1 to 5% w/w of an additional active agent.

4.3. Pharmaceutical Dosage forms

As summarized above, this disclosure provides a pharmaceutical dosage form comprising the amorphous solid dispersion (ASD) or pharmaceutical composition described herein. The disclosure provides for tablets, pills, capsules and the like, comprising the amorphous solid dispersion (ASD) or pharmaceutical compositions or dosage forms described herein. Accordingly, in some embodiments, the pharmaceutical dosage from is a solid dosage form. In certain cases, the solid dosage form is a capsule. In certain cases, the capsule is an opaque hard gelatin capsule. In certain cases, the dosage form is a tablet.

In an embodiment, pharmaceutical compositions comprising amorphous solid dispersion of ebselen, and pharmaceutically acceptable excipients are prepared by using, but not limited, to wet granulation, dry granulation, and direct compression.

In an embodiment, pharmaceutical compositions comprising amorphous solid dispersion of ebselen, and pharmaceutically acceptable excipients are prepared by using direct compression, which process comprises mixing amorphous solid dispersion of ebselen and pharmaceutically acceptable excipients, the resultant mixture is either compressed to tablet or filled in hard gelatin capsules.

In an embodiment, pharmaceutical compositions comprising amorphous solid dispersion of ebselen, and pharmaceutically acceptable excipients are prepared by using dry granulation, wherein dry granulation is carried out by either direct compaction or roller compaction or both.

In an embodiment, pharmaceutical compositions comprising amorphous solid dispersion of ebselen, and pharmaceutically acceptable excipients are prepared by using direct compaction dry granulation, which process comprises compressing mixture of amorphous solid dispersion of ebselen and intragranular material into slug, compressed slugs are milled and passed through mess screen manually or automatically which results in granules. The resulting granules were mixed with extra-granular material. This final mixture is either compressed to tablet or filled in hard gelatin capsules.

In an embodiment, pharmaceutical compositions comprising amorphous solid dispersion of ebselen and pharmaceutically acceptable excipients are prepared by using roller compaction dry granulation, which process comprises passing a mixture of amorphous solid dispersion of ebselen and intragranular material between two high-pressure rollers to form consolidated and densified material, the resultant densified material is then reduced to a uniform granule size by milling, which were then mixed with extra-granular material. This final mixture is either compressed to tablet or filled in hard gelatin capsules.

In an embodiment, pharmaceutical compositions comprising amorphous solid dispersion of ebselen and pharmaceutically acceptable excipients are prepared by wet granulation, which process comprises: (a) mixing amorphous solid dispersion of ebselen and pharmaceutically acceptable excipients (b) adding sufficient solvent to the mixture obtained from step (a) under shear to generate granules; (c) milling or grinding the granules followed by sieving of said granules; optionally mixing with other excipients. This final mixture is either compressed to tablet or filled in hard gelatin capsules.

The disclosure also relates to compositions comprising amorphous solid dispersion of ebselen for oral administration, which solid dispersion further comprises at least one carrier polymer, which composition comprises (a) an immediate-release portion and (b) a modified-release portion.

As used herein the term “immediate-release” refers to the rapid release of the majority of the therapeutic compound. Particularly useful conditions for immediate-release are release of at least or equal to about 80% of the therapeutic compound within thirty minutes after oral ingestion. The particular immediate-release conditions for a specific therapeutic compound will be recognized or known by one of ordinary skill in the art.

As used herein the term “modified-release” refers to slower release of the majority of the therapeutic compound as compared to immediate release dosage forms. The particular modified-release conditions for a specific therapeutic compound will be recognized or known by one of ordinary skill in the art.

The compositions are manufactured by processes such as direct compression, wet granulation or dry granulation. The pharmaceutical compositions are in the form of oral dosage forms, preferably solid oral dosage forms, including capsules, tablets and multi-particulates.

Physically, the combination of active ingredient and vehicle may either form a solid dispersion, i.e., the active ingredient is dispersed in the vehicle in particulate form, or may form a solid solution, i.e., the active ingredient is dissolved in the vehicle at a molecular level. The active ingredient and the vehicle may also form a solid dispersion having therein a part of the active ingredient dissolved at a molecular level. The physical state of the dispersion and/or solution may be determined by using various techniques such as Hot Stage Microscopy (HSM), Differential Scanning calorimetry (DSC), Scanning Electron Microscopy (SEM) optionally in combination with Energy Dispersive X-ray (EDX), and X-ray powder diffraction.

In some embodiment, the amorphous solid dispersions of ebselen of the present disclosure comprise ebselen, and at least one carrier polymer, wherein the carrier polymer is a copovidone polymer. In some embodiments, ebselen and the copovidone polymer are present in a ratio of 1:4 (w/w).

In an embodiment, the obtained amorphous solid dispersions of ebselen are combined with other pharmaceutically acceptable excipients such as diluents, binders, disintegrating agents, glidants, lubricants, plasticizers, colorants and surfactants to make it into finished dosage form (e.g., as described herein). The pharmaceutical compositions comprising the subject amorphous solid dispersions of ebselen, and pharmaceutically acceptable excipients are prepared by using processes such as wet granulation, dry granulation, or direct compression. The dry granulation process may be carried out either by direct compaction or roller compaction or both.

The pharmaceutical compositions comprising amorphous solid dispersions of ebselen, and pharmaceutically acceptable excipients can be prepared by using direct compaction dry granulation, which process comprises of compressing mixture of the amorphous solid dispersions of ebselen and intra-granular excipients into slug; compressed slugs are milled and passed through mess screen manually or automatically. The resulting granules are mixed with extra-granular excipients. This final mixture is either compressed into tablet or filled in capsules.

The pharmaceutical compositions comprising amorphous solid dispersions of ebselen and pharmaceutically acceptable excipients may also be prepared by using roller compaction dry granulation, which process comprises passing mixture of solid dispersion and intra-granular excipients between two high-pressure rollers to form consolidated and densified material; the resultant densified material is then reduced to a uniform granule size by milling, which are then mixed with extra-granular excipients. This final mixture is either compressed into tablet or filled in capsules.

The pharmaceutical compositions comprising amorphous solid dispersions of ebselen, and pharmaceutically acceptable excipients may also be prepared by using direct compression, which process comprises of mixing the amorphous solid dispersions of ebselen and pharmaceutically acceptable excipients; the resultant mixture is either compressed into tablets or filled in capsules.

The pharmaceutical compositions comprising amorphous solid dispersions of ebselen and pharmaceutically acceptable excipients can also be prepared by wet granulation, which process comprises of: (a) mixing amorphous solid dispersions of ebselen and pharmaceutically acceptable excipients (b) adding sufficient solvent to the mixture obtained from step (a) under shear to generate granules; (c) milling or grinding the granules followed by sieving of said granules; optionally mixing with other excipients. This final mixture is either compressed into tablet or filled in capsules.

In some embodiments, he pharmaceutical composition of the present disclosure is a granulate/particulate material. The granules/particles may be filled into a capsule or compressed into a tablet. The tablet may optionally be coated with an additional enteric polymer or an immediate-release coating.

Moreover, the extrudates/granules of the present disclosure may be formulated into any suitable dosage form, including but not limited to oral suspensions, gels, tablets, capsules, immediate release formulations, delayed release formulations, controlled release formulations, extended-release formulations, pulsatile release formulations, and mixed immediate and controlled release formulations.

The tablets or pills of the present disclosure may be coated to provide a dosage form affording the advantage of prolonged action or to protect from the acid conditions of the stomach. The tablets may also be formulated for immediate release. In certain embodiments, the tablet comprises a film coating. A film coating may be useful for limiting photolytic degradation. Suitable film coatings are selected by routine screening of commercially available preparations. In one embodiment, the film coating is a hypromellose-based coating. In certain embodiments, the coating comprises a film-forming agent, a plasticizer, a glidant and optionally one or more pigments. An exemplary film coating composition may comprise hydroxypropyl methylcellulose (HPMC), lactose monohydrate, titanium dioxide, and triglyceride 1,2,3-triacetoxypropane (triacetin). In certain cases, the film coating composition may comprise hydroxypropyl methylcellulose (HPMC), polyethylene glycol (PEG), talc, titanium dioxide and optionally iron oxide, including iron oxide red and/or yellow.

In certain embodiments, the pharmaceutical dosage form comprises the ebselen in a therapeutically effective amount (e.g., as described herein for ebselen).

In certain embodiments, the pharmaceutical dosage form comprises from about 45% to about 60% w/w ebselen. In some embodiments, the pharmaceutical dosage form comprises from about 50% to about 60% w/w ebselen. In some embodiments, the pharmaceutical dosage form comprises about 50% w/w, about 51% w/w, about 52% w/w, about 53% w/w, about 54% w/w, about 55% w/w, about 56% w/w, about 57% w/w, about 58% w/w, about 59% w/w, or about 60% w/w ebselen. In a specific embodiment, the pharmaceutical dosage form comprises about 55-58% w/w ebselen.

In certain embodiments, the pharmaceutical dosage form comprises from 45% to 60% w/w ebselen. In some embodiments, the pharmaceutical dosage form comprises from 50% to 60% w/w ebselen. In some embodiments, the pharmaceutical dosage form comprises 50% w/w, 51% w/w, 52% w/w, 53% w/w, 54% w/w, 55% w/w, 56% w/w, 57% w/w, 58% w/w, 59% w/w, or 60% w/w ebselen. In a specific embodiment, the pharmaceutical dosage form comprises 55-58% w/w ebselen.

In some embodiments, the pharmaceutical dosage form comprises from about 50 mg to about 400 mg ebselen. In some embodiments, the pharmaceutical dosage form comprises from about 50 mg to about 100 mg ebselen, or from about 100 mg to about 150 mg ebselen, or from about 150 mg to about 200 mg ebselen, or from about 250 mg to about 300 mg ebselen, or from about 300 mg to about 350 mg, or from about 350 mg to about 400 mg. In further embodiments, the pharmaceutical dosage form comprises about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg ebselen. In further embodiments, the pharmaceutical dosage form comprises about 210 mg, about 220 mg, about 230, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg ebselen. In a specific embodiment, the pharmaceutical dosage form comprises about 50 mg ebselen. In a specific embodiment, the pharmaceutical dosage form comprises about 100 mg ebselen. In some embodiments, the pharmaceutical dosage form comprises about 200 mg ebselen. In some embodiments, the pharmaceutical dosage form comprises about 400 mg ebselen.

In some embodiments, the pharmaceutical dosage form comprises from 50 mg to 400 mg ebselen. In some embodiments, the pharmaceutical dosage form comprises from 50 mg to 100 mg ebselen, or from 100 mg to 150 mg ebselen, or from 150 mg to 200 mg ebselen, or from 250 mg to 300 mg ebselen, or from 300 mg to 350 mg, or from 350 mg to 400 mg. In further embodiments, the pharmaceutical dosage form comprises 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg ebselen. In further embodiments, the pharmaceutical dosage form comprises 210 mg, 220 mg, 230, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, or 400 mg ebselen. In a specific embodiment, the pharmaceutical dosage form comprises 50 mg ebselen. In a specific embodiment, the pharmaceutical dosage form comprises 100 mg ebselen. In some embodiments, the pharmaceutical dosage form comprises 200 mg ebselen. In some embodiments, the pharmaceutical dosage form comprises 400 mg ebselen.

In certain embodiments, the pharmaceutical dosage form comprises one or more excipients (e.g., as described herein). In certain embodiments, the pharmaceutical dosage form comprises one or more fillers. In certain embodiments, the pharmaceutical dosage from comprises microcrystalline cellulose.

In certain embodiments, the pharmaceutical dosage form comprises microcrystalline cellulose in an amount from about 140 mg to 280 mg, such as 145 mg to 275 mg, 145 mg to 270 mg, 145 mg to 265 mg, or 150 to 265 mg. In a specific embodiment, the microcrystalline cellulose is in an amount of about 265 mg.

In certain embodiments, the pharmaceutical dosage form comprises microcrystalline cellulose in an amount from 140 mg to 280 mg, such as 145 mg to 275 mg, 145 mg to 270 mg, 145 mg to 265 mg, or 150 to 265 mg. In a specific embodiment, the microcrystalline cellulose is in an amount of 265 mg.

In certain embodiments, the pharmaceutical dosage form comprises one or more disintegrants. In certain cases, the disintegrant is croscarmellose sodium. In certain embodiments, the pharmaceutical dosage form comprises croscarmellose sodium in an amount from about 14 mg to 65 mg, such as about 14 mg to 60 mg, 14 mg to 50 mg, 14 mg to 45 mg, 14 mg to 40 mg, 14 to 35 mg, or about 14 to 30 mg. In certain embodiments, the pharmaceutical dosage from comprises croscarmellose sodium in an amount of from about 20 mg to 40 mg, such as about 20 mg to 35 mg, about 20 mg to 30 mg, or about 25 mg to 30 mg. In a specific embodiment, the croscarmellose sodium is in an amount of about 28 mg.

In certain embodiments, the pharmaceutical dosage form comprises croscarmellose sodium in an amount from 14 mg to 65 mg, such as 14 mg to 60 mg, 14 mg to 50 mg, 14 mg to 45 mg, 14 mg to 40 mg, 14 to 35 mg, or 14 to 30 mg. In certain embodiments, the pharmaceutical dosage from comprises croscarmellose sodium in an amount of from 20 mg to 40 mg, such as 20 mg to 35 mg, 20 mg to 30 mg, or 25 mg to 30 mg. In a specific embodiment, the croscarmellose sodium is in an amount of 28 mg.

In certain embodiments, the pharmaceutical dosage form comprises one or more lubricants. In certain cases, the lubricant is magnesium stearate. In certain embodiments, the pharmaceutical dosage form comprises magnesium stearate in an amount from about 0.7 mg to 7 mg, such as about 0.7 mg to 6 mg, about 0.7 mg to 5 mg, about 0.7 mg to 4 mg, or about 0.7 to 3.5 mg. In certain embodiments, the pharmaceutical dosage from comprises magnesium stearate in an amount from about 1.5 mg to 5 mg, such as about 1.5 to 4 mg, about 2 mg to 4 mg, about 2.5 mg to 4 mg, or about 3 mg to 4 mg. In a specific embodiment, the magnesium stearate is in an amount of about 3.5 mg.

In certain embodiments, the pharmaceutical dosage form comprises magnesium stearate in an amount from 0.7 mg to 7 mg, such as 0.7 mg to 6 mg, 0.7 mg to 5 mg, 0.7 mg to 4 mg, or 0.7 to 3.5 mg. In certain embodiments, the pharmaceutical dosage from comprises magnesium stearate in an amount from 1.5 mg to 5 mg, such as 1.5 to 4 mg, 2 mg to 4 mg, 2.5 mg to 4 mg, or 3 mg to 4 mg. In a specific embodiment, the magnesium stearate is in an amount of 3.5 mg.

In certain embodiments, the pharmaceutical dosage form comprises one or more glidants. In certain cases, the glidant is colloidal silicon dioxide. In certain embodiments, the pharmaceutical dosage form comprises colloidal silicon dioxide in an amount from about 0.7 mg to 7 mg, such as about 0.7 mg to 6 mg, 0.7 mg to 5 mg, 0.7 mg to 4 mg, or about 0.7 to 3.5 mg. In certain embodiments, the pharmaceutical dosage from comprises colloidal silicon dioxide in an amount from about 1.5 mg to 5 mg, such as about 1.5 to 4 mg, about 2 mg to 4 mg, about 2.5 mg to 4 mg, or about 3 mg to 4 mg. In a specific embodiment, the colloidal silicon dioxide is in an amount of about 3.5 mg.

In certain embodiments, the pharmaceutical dosage form comprises colloidal silicon dioxide in an amount from 0.7 mg to 7 mg, such as 0.7 mg to 6 mg, 0.7 mg to 5 mg, 0.7 mg to 4 mg, or 0.7 to 3.5 mg. In certain embodiments, the pharmaceutical dosage form comprises colloidal silicon dioxide in an amount from 1.5 mg to 5 mg, such as 1.5 to 4 mg, 2 to 4 mg, 2.5 mg to 4 mg, or 3 mg to 4 mg. In a specific embodiment, the colloidal silicon dioxide is in an amount of 3.5 mg.

In certain embodiments, the pharmaceutical dosage from comprises a) 200-400 mg ebselen; b) 140-280 mg microcrystalline cellulose; c) 14 to 63 mg croscarmellose sodium; d) 0.7-7 mg magnesium stearate; and 0.7-7 mg colloidal silicon dioxide.

In certain embodiments, the pharmaceutical dosage from comprises a) 50 mg ebselen amorphous spray-dried dispersion in hot melt extrusion of copovidone in 0.5% methyl cellulose and 0.2% polyethylene sorbitol ester.

In some embodiments, the subject pharmaceutical dosage form (e.g., as described herein) may be administered orally. In certain cases, the pharmaceutical dosage form (e.g., as described herein) is administered to a human subject to produce: a maximum blood plasma concentration (average Cₘₐₓ) that is 2-fold greater than that achieved with a control ebselen formulation (e.g., as described herein); and/or an area under the curve (AUC) for ebselen and/or an ebselen metabolite that is at least 2-fold greater than that achieved with a control ebselen formulation. In certain cases, the ebselen metabolite is ebselen glucuronide.

In some embodiments, the pharmaceutical composition, pharmaceutical dosage form, or tablet as described herein is free of negative drug-drug interactions. In a related embodiment, the pharmaceutical composition, pharmaceutical dosage form, or tablet is free of negative drug-drug interactions with other active agents. In a further embodiment, the pharmaceutical composition, pharmaceutical dosage form, or tablet as described herein is administrable without regard to food and with or without regard to the patient being on another therapeutic agent.

### 4.4. Methods of use

As summarized above, also provided herein are methods of delivering the subject ebselen pharmaceutical dosage forms to a subject to achieve an enhanced maximum blood plasma concentration (Cₘₐₓ) for ebselen that is at least 2-fold greater than that achieved with a control ebselen formulation (e.g., as described herein, and/or an area under the curve (AUC) for ebselen and/or an ebselen metabolite that is at least 2-fold greater than that achieved with a control ebselen formulation. In certain cases, the ebselen metabolite is ebselen glucuronide.

An exemplary dosage may be a solid dosage from (e.g., a tablet) taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release. In certain embodiments, the pharmaceutical dosage form is administered twice daily. In some embodiments, the pharmaceutical dosage form is administered three times daily.

In some embodiments, multiple doses of the pharmaceutical dosage form are administered. The frequency of administration of the dosage form can vary depending on any of a variety of factors, e.g., severity of the symptoms, etc. For example, in some embodiments, the subject dosage form is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid). In certain embodiments, the subject dosage form is administered twice a day (bid).

The duration of administration of a pharmaceutical dosage from, e.g., the period of time over which ebselen is administered, can vary, depending on any of a variety of factors, e.g., patient response, etc. For example, an active agent can be administered over a period of time ranging from about one day to about one week, from about two weeks to about four weeks, from about one month to about two months, or from about two months to about four months, or more.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is in some embodiments one which provides a Cₘₐₓ of about 100 ng/mL to about 2500 ng/ml of ebselen in a blood sample taken from the individual being treated, after administration of the subject dosage form to the individual.

In some embodiments, the subject pharmaceutical dosage form is administered in an amount that provides, a mean blood or plasma Cₘₐₓ of ebselen or at least 100 ng/ml, 150 ng/ml, 200 ng/ml, 250 ng/ml, 300 ng/ml, 350 ng/mL, 400 ng/ml, 450 ng/ml, 500 ng/ml, 550 ng/mL, 600 ng/mL, 650 ng/mL, 700 ng/mL, 750 ng/mL, 800 ng/ml, 850 ng/ml, 900 ng/mL, 950 ng/mL, 1000 ng/mL, 1050 ng/mL, 1100 ng/mL, 1150 ng/mL, 1200 ng/ml, 1250 ng/mL, 1300 ng/mL, 1350 ng/mL, 1400 ng/mL, 1450 ng/mL, 1500 ng/mL, 1550 ng/mL, 1600 ng/mL, 1650 ng/ml, 1700 ng/mL, 1750 ng/mL, 1800 ng/mL, 1850 ng/ml, 1900 ng/mL, 1950 ng/mL, 2000 ng/mL, 2250 ng/mL, or at least 2500 ng/mL.

In some embodiments, the subject pharmaceutical dosage form is administered in an amount that provides, within 1 day following administration, a mean blood or plasma Cₘₐₓ of ebselen that is at least 2-fold greater than the Cₘₐₓ achieved with the subject control ebselen formulation (e.g., as described herein in Example 1). In some cases, the maximum blood plasma concentration (Cₘₐₓ) achieved after one (1) day is at least about 2.1-fold greater than the Cₘₐₓ achieved with a control ebselen formulation, e.g., at least about 2.2-fold, 2.3-fold, 2.4-fold, 2.5-fold, 3-fold, 3.5-fold, 4-fold, 4.5-fold, 5-fold or 5.5-fold greater than the Cₘₐₓ achieved with a control ebselen formulation.

In some embodiments, the subject pharmaceutical dosage form is administered in an amount that provides, an area under the curve (AUC) for ebselen that is at least 2-fold greater than the AUC achieved with the subject control ebselen formulation (e.g., as described herein in Example 1). In some cases, the AUC achieved for ebselen is at least about 2.1-fold greater than the AUC achieved for ebselen with a control ebselen formulation, e.g., at least about 2.2-fold, 2.3-fold, 2.4-fold, 2.5-fold, 3-fold, 3.5-fold, 4-fold, 4.5-fold, 5-fold or 5.5-fold greater than the AUC achieved with a control ebselen formulation.

In some embodiments, the subject pharmaceutical dosage form is administered in an amount that provides, an area under the curve (AUC) for an ebselen metabolite that is at least 2-fold greater than the AUC achieved with the subject control ebselen formulation (e.g., as described herein in Example 1). In some cases, the AUC achieved for the ebselen metabolite is at least about 2.1-fold greater than the Cₘₐₓ achieved with a control ebselen formulation, e.g., at least about 2.2-fold, 2.3-fold, 2.4-fold, 2.5-fold, 3-fold, 3.5-fold, 4-fold, 4.5-fold, 5-fold or 5.5-fold greater than the AUC achieved for the ebselen metabolite with a control ebselen formulation. In some embodiments, the ebselen metabolite is ebselen glucuronide.

In some embodiments, the subject pharmaceutical dosage form administered as an amorphous form of ebselen exhibits greater (e.g., 2-fold greater or more) mean peak concentration (Cₘₐₓ) as compared to a dosage form administered as two dry blend capsules each comprising 200 mg ebselen, 120 mg microcrystalline cellulose, 28 mg croscarmellose sodium and 1.8 mg magnesium stearate. In certain cases, the pharmaceutical dosage form administered as an amorphous form of ebselen exhibits at least a 2.0-fold, a 2.5-fold, a 3-fold, or at least a 3.5-fold greater mean peak concentration (Cₘₐₓ) as compared to a dosage form administered as two capsules each comprising 200 mg ebselen, 120 mg microcrystalline cellulose, 28 mg croscarmellose sodium and 1.8 mg magnesium stearate.

In some embodiments, the subject pharmaceutical dosage form administered as an amorphous form of ebselen exhibits greater (e.g., 2-fold greater or more) AUC for ebselen as compared to a dosage form administered as two dry blend capsules each comprising 200 mg ebselen, 120 mg microcrystalline cellulose, 28 mg croscarmellose sodium and 1.8 mg magnesium stearate. In certain cases, the pharmaceutical dosage form administered as an amorphous form of ebselen exhibits at least a 2.0-fold, a 2.5-fold, a 3-fold, or at least a 3.5-fold greater AUC for ebselen as compared to a dosage form administered as two capsules each comprising 200 mg ebselen, 120 mg microcrystalline cellulose, 28 mg croscarmellose sodium and 1.8 mg magnesium stearate.

In some embodiments, the subject pharmaceutical dosage form administered as an amorphous form of ebselen exhibits greater (e.g., 2-fold greater or more) AUC for an ebselen metabolite as compared to a dosage form administered as two dry blend capsules each comprising 200 mg ebselen, 120 mg microcrystalline cellulose, 28 mg croscarmellose sodium and 1.8 mg magnesium stearate. In certain cases, the pharmaceutical dosage form administered as an amorphous form of ebselen exhibits at least a 2.0-fold, a 2.5-fold, a 3-fold, or at least a 3.5-fold greater AUC for an ebselen metabolite as compared to a dosage form administered as two capsules each comprising 200 mg ebselen, 120 mg microcrystalline cellulose, 28 mg croscarmellose sodium and 1.8 mg magnesium stearate. In some embodiments, the ebselen metabolite is ebselen glucuronide.

Depending on the subject and condition being treated and on the administration route, the pharmaceutical dosage form may be administered with ebselen dosages of, for example, 0.5 to 150 mg/kg body weight per day, e.g., from about 0.5 mg/kg body weight per day to about 1 mg/kg body weight per day, from about 1 mg/kg body weight per day to about 25 mg/kg body weight per day, from about 25 mg/kg body weight per day to about 50 mg/kg body weight per day, from about 50 mg/kg body weight per day to about 100 mg/kg body weight per day, from about 100 mg/kg body weight per day to about 125 mg/kg body weight per day, from about 125 mg/kg body weight per day to about 150 mg/kg body weight per day. The range is broad, since in general the efficacy of a therapeutic effect of ebselen for different mammals varies widely with doses typically being 20, 30 or even 40 times smaller (per unit body weight) in man than in the rat. Similarly, the mode of administration can have a large effect on dosage. Thus, for example, oral dosages may be about ten times the injection dose. Higher doses may be used for localized routes of delivery.

Depending on the subject and condition being treated and on the administration route, the pharmaceutical dosage form may be administered with ebselen dosages of, for example, 50 to 10,000 mg/dose, e.g., from about 50 mg/dose to about 100 mg/dose, from about 100 mg/dose to about 200 mg/dose, from about 200 mg/dose to about 250 mg/dose, from about 300 mg/dose to about 350 mg/dose, from about 350 mg/dose to about 400 mg/dose, from about 400 mg/dose to about 450 mg/dose, from about 450 mg/dose to about 500 mg/dose, form about 500 mg/dose to about 550 mg/dose, from about 600 mg/dose to about 650 mg/dose, from about 650 mg/dose to about 700 mg/dose, from about 700 mg/dose to about 750 mg/dose, from about 750 mg/dose to about 800 mg/dose, from about 800 mg/dose to about 850 mg/dose, from about 850 mg/dose to about 900 mg/dose, from about 900 mg/dose to about 950 mg/dose, from about 950 mg/dose to about 1000 mg/dose, from about 1,000 mg/dose to about 2,500 mg/dose, from about 2,500 mg/dose to about 5,000 mg/dose, from about 5,000 mg/dose to about 7500 mg/dose, from about 7,500 mg/dose to about 10,000 mg/dose. In some embodiments, the pharmaceutical dosage form may be administered in dosages of, for example, 0.1 to 10 g/dose, e.g., from about 0.1 g/dose to 0.25 g/dose, from about 0.2 g/dose to 0.4 g/dose, from about 0.4 g/dose to 0.5 g/dose, from about 0.5 g/dose to 1 g/dose, from about 1g/dose to 3 g/dose, from about 3 g/dose to 5 g/dose, from about 5 g/dose to 6 g/dose, from about 6 g/dose to about 8 g/dose, from about 8 g/dose to about 10 g/dose.

In some embodiments, the pharmaceutical dosage form comprises 50 mg ebselen, and is administered orally (e.g., in a tablet) twice daily.

In some embodiments, the pharmaceutical dosage form comprises 100 mg ebselen, and is administered orally (e.g., in a tablet) twice daily.

In some embodiments, the pharmaceutical dosage form comprises 200 mg ebselen, and is administered orally (e.g., in a tablet) twice daily.

In some embodiments, the pharmaceutical dosage form comprises 400 mg ebselen, and is administered orally (e.g., in a tablet) twice daily.

### 4.4.1. Therapeutic Indications

Aspects of the present disclosure include methods of treating therapeutic indications of interest using the subject pharmaceutical compositions or dosage forms comprising ebselen (e.g., as described herein). The term “therapeutic indication” refers to any symptom, condition, disorder, or disease that may be alleviated, stabilized, improved, cured, or otherwise addressed by some form of treatment or other therapeutic intervention with ebselen. In some embodiments, methods of the present disclosure may include treating ebselen-related indications by administering compositions or pharmaceutical dosage forms disclosed herein (e.g., dosage forms comprising ebselen).

Ebselen has been shown to have use in the treatment and/or prevention of a range of diseases and conditions. Examples include but are not limited to, noise-induced hearing loss, Meniere's disease, chemotherapy induced ototoxicity, aminoglycoside-induced ototoxicity in patients with cystic fibrosis, and COVID-19.

### 4.5. Kits

Also provided herein is a pharmaceutical kit comprising a package containing a plurality of unit pharmaceutical dosage forms (e.g., as described herein) and instructions for use.

In accordance with embodiments of the invention, the pharmaceutical kit comprises a container, such as a high-density polyethylene (HDPE) bottles, or a box including one or more blister packs, wherein the bottles or blister packs can contain a plurality of solid unit pharmaceutical dosage forms as described herein. In certain embodiments, the container or pack comprises at least 5, at least 8, at least 10, at least 12 of at least 15 of said unit pharmaceutical dosage forms, e.g., 4, 5, 6, 7, 8, 9, 10, 11, 12, 13, 14, 15, 16, 17, 18, 19 or 20 of said unit dosage forms.

In accordance with the invention, the pharmaceutical kit comprises instructions (e.g., a leaflet) inserted into the container or box, typically a patient information leaflet containing printed information, which information may include a description of the form and composition of the unit pharmaceutical dosage forms contained in the kit, an indication of the therapeutic indications for which the product is intended, instructions as to how the product is to be used and information and warnings concerning adverse effects and contraindications associated with the use. In accordance with the present disclosure, the leaflet will usually contain the information concerning the therapeutic indications, uses, treatment regimens, etc. as described herein in relation to the methods of treatment of the present invention. In certain cases, the leaflet contains printed instructions to repeatedly (self-) administer the pharmaceutical unit dosage forms in order to treat and/or prevent a disease or condition, such as Meniere's Disease, conditions related to noise- or ototoxin-induced injury, acute lung infections and related conditions or diseases, such as coronavirus-mediated lung injuries.

### 4.6. Definitions

As used herein, the term “solid dispersion” is meant a molecular dispersion of a compound, particularly a drug substance within a carrier. The term solid dispersion in general means a system in solid state comprising at least two components, wherein one component is dispersed substantially evenly throughout the other component(s). For example, solid dispersions may be the dispersion of one or more active ingredients in an inert carrier or matrix at solid state, prepared by the melting, solvent, or melting-solvent methods. While not wishing to be bound by theory, in a solid dispersion, the drug may be present in a molecular state, colloidal state, metastable state, or an amorphous state. Formation of a molecular dispersion may provide a means of reducing the particle size to nearly molecular levels (i.e., there are no particles).

As used herein, the term “otoprotectant” refers to a chemical substance that is capable of ameliorating hearing loss.

As used herein, the term “ameliorating hearing loss” includes: (a) reducing the magnitude and/or duration of hearing loss; and/or (b) slowing the progression of hearing loss; and/or (c) preventing the onset of hearing loss that would occur without administration of an otoprotectant composition of the invention.

As used herein, the term “ototoxic agent” means an agent that is likely to impair the function of any component of the ear involved in hearing and, therefore, is likely to induce temporary or permanent hearing loss. Examples of ototoxic agents are ototoxic drugs and ototoxic noise.

As used herein, the term “exposure to an ototoxic agent” includes single or multiple exposures to an ototoxic agent that is recognized in the art as being likely to cause temporary or permanent hearing loss. For example, the Occupational Safety and Health Administration (OSHA) considers exposures to noise greater than or equal to 85 decibels (dB) to be hazardous to hearing. Thus, OSHA mandates that workers not be exposed to greater than or equal to 85 dB of noise over a continuous eight hour period based on a time weighted average, unless noise reduction measures (i.e., earmuffs) are employed.

As used herein, the term “otoprotectant composition” refers to a composition that includes at least one otoprotectant, and may include more than one otoprotectant. Otoprotectant compositions may also include, in addition to one or more otoprotectant(s), pharmaceutically acceptable carriers that facilitate administration of an otoprotectant composition to a mammalian subject.

The abbreviation “mg” means milligrams.

The abbreviation “BID” means “twice daily.”

The abbreviation “QD” means “once daily.”

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” are used in their broadest sense understood in the clinical arts.

The term “pharmaceutically acceptable” indicates that the material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

The term “carrier” refers to a glidant, diluent, adjuvant, excipient, or vehicle etc. with which the compound is administered, without limitation. Examples of carriers are described herein and also in Remington: The Science and Practice of Pharmacy (Remington: The Science and Practice of Pharmacy, 23rd Edition, ISBN-13:978-0128200070).

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropyl cellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, modified corn starch, sodium carboxymethyl starch, crospovidone, pregelatinized starch, and alginic acid.

The term “lubricant” refers to an excipient which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, calcium stearate, polyethylene glycol, sodium stearyl fumarate, or talc; and solubilizers such as fatty acids including lauric acid, oleic acid, and C8/C10 fatty acid.

The term “film coating” refers to a thin, uniform, film on the surface of a substrate (e.g., tablet). Film coatings are particularly useful for protecting the active ingredient from photolytic degradation. Non-limiting examples of film coatings include polyvinylalcohol based, hydroxyethyl cellulose, hydroxypropylmethylcellulose, sodium carboxymethylcellulose, polyethylene glycol 4000 and cellulose acetate phthalate film coatings.

The term “glidant” as used herein is intended to mean agents used in tablet and capsule formulations to improve flow-properties during tablet compression and to produce an anti-caking effect. Non-limiting examples of glidants include colloidal silicon dioxide, talc, fumed silica, starch, starch derivatives, and bentonite.

The term “effective amount” or “therapeutically effective amount” refers to an amount that is sufficient to effect treatment, as defined herein, when administered to a mammal in need of such treatment. The therapeutically effective amount will vary depending upon the patient being treated, the weight and age of the patient, the severity of the disease condition, the manner of administration and the like, which can readily be determined by one of ordinary skill in the art.

The term “unit dosage forms” or “pharmaceutical dosage forms” refers to physically discrete units suitable as unitary dosages for human patients and other mammals, each unit containing a predetermined quantity of active material calculated to produce the desired therapeutic effect, in association with a suitable pharmaceutical excipient (e.g., a tablet).

The term “treatment” or “treating,” to the extent it relates to a disease or condition includes preventing the disease or condition from occurring, inhibiting the disease or condition, eliminating the disease or condition, and/or relieving one or more symptoms of the disease or condition.

The term “% w/w” as used herein refers to the weight of a component based on the total weight of a composition comprising the component. For example, if component A is present in an amount of 50% w/w in a 100 mg composition, component A is present in an amount of 50 mg.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or(S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its(S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. Unless otherwise specified, the term “about” means within plus or minus 10% of a the explicitly recited value, rounded either up or down to the nearest integer.

### 5. Additional Embodiments

The disclosure is further described by the following non-limiting clauses:

Clause 1. An amorphous solid dispersion (ASD) comprising an amorphous form of ebselen and a carrier polymer.

Clause 2. The amorphous solid dispersion of clause 1, comprising 10-30% w/w of ebselen.

Clause 3. The amorphous solid dispersion of clause 2, comprising 15-25% w/w of ebselen.

Clause 4. The amorphous solid dispersion of clause 3, comprising about 20% w/w ebselen.

Clause 5. The amorphous solid dispersion of any one of clauses 1 to 4, wherein the carrier polymer is selected from a polyvinylpyrrolidone polymer, a copovidone polymer, a hydroxypropyl methyl cellulose polymer, a polyvinyl caprolactam-polyvinyl acetate-polyethylene glycol graft co-polymer, a dimethylaminoethyl methacrylate-copolymer, a methacrylic acid-methyl methacrylate copolymer, and a polyethylene glycol polymer.

Clause 6. The amorphous solid dispersion of clause 5, wherein the carrier polymer is a copovidone polymer, a hydroxypropyl methyl cellulose polymer, a polyvinyl caprolactam-polyvinyl acetate-polyethylene glycol graft co-polymer, or a combination thereof.

Clause 7. The amorphous solid dispersion of clause 6, wherein the carrier polymer is a copovidone polymer.

Clause 8. The amorphous solid dispersion of any one of clauses 1 to 7, wherein the solid dispersion is formed by hot melt extrusion of a mixture comprising ebselen and a carrier protein.

Clause 9. A pharmaceutical composition comprising the amorphous solid dispersion of any one of clauses 1 to 8 and one or more pharmaceutically acceptable excipients.

Clause 10. The pharmaceutical composition of clause 9, wherein the one or more excipients comprises a thickener, a solubilizer, a surfactant an emulsifier, or a combination thereof.

Clause 11. The pharmaceutical composition of clause 9 or 10, further comprising methyl cellulose.

Clause 12. The pharmaceutical composition of any one of clauses 9 to 11, further comprising polyethylene sorbitol ester.

Clause 13. The pharmaceutical composition of any one of clauses 9 to 12, further comprising a mixture of methyl cellulose and polyethylene sorbitol ester.

Clause 14. A pharmaceutical dosage form comprising the amorphous solid dispersion of any one of clauses 1 to 8, or a pharmaceutical composition of any one of clauses 9 to 13.

Clause 15. The pharmaceutical dosage form of clause 14, in the form of a powder, rod, tablet, capsule, or any other form that results from spray drying, hot melt extrusion or high shear mixing of the amorphous solid dispersion.

Clause 16. The pharmaceutical dosage form of clause 14 or 15, wherein the ebselen is present in an amount of from 50 mg to 200 mg.

Clause 17. The pharmaceutical dosage form of clause 16, wherein the ebselen is present in an amount of about 50 mg.

Clause 18. The pharmaceutical dosage form of clause 16, wherein the ebselen is present in an amount of about 100 mg.

Clause 19. The pharmaceutical dosage form of clause 16, wherein the ebselen is present in an amount of about 200 mg.

Clause 20. The pharmaceutical dosage form of clause 16, wherein the ebselen is present in an amount of about 400 mg.

Clause 21. The pharmaceutical dosage form of clause 14, wherein the dosage form comprises 50 mg ebselen amorphous spray-dried dispersion in hot melt extrusion of copovidone 79.3%, 0.5% methyl cellulose and 0.2% polyethylene sorbitol ester.

Clause 22. The pharmaceutical dosage form of clause 21, wherein the dosage form is a capsule.

Clause 23. The pharmaceutical dosage form of any one of clauses 14 to 22, wherein oral administration of said pharmaceutical dosage form to a selected human subject group produces in said selected human subject group:
an enhanced maximum blood plasma concentration (Cₘₐₓ) for ebselen that is at least 2-fold greater than that achieved with a control ebselen formulation;
an area under the curve (AUC) for ebselen that is at least 2-fold greater than that achieved with a control ebselen formulation; and
an area under the curve (AUC) for ebselen metabolite that is at least 2-fold greater than that achieved with a control ebselen formulation.

Clause 24. A method of delivering a therapeutically effective amount of ebselen to a subject in need thereof, comprising orally administering to a subject in need thereof a pharmaceutical dosage form according to any one of clauses 14 to 23, to achieve:
an enhanced maximum blood plasma concentration (Cₘₐₓ) for ebselen that is at least 2-fold greater than that achieved with a control ebselen formulation;
an area under the curve (AUC) for ebselen that is at least 2-fold greater than that achieved with a control ebselen formulation.
an area under the curve (AUC) for ebselen metabolite that is at least 2-fold greater than that achieved with a control ebselen formulation.

Clause 25. The method of clause 24, wherein the pharmaceutical dosage form is administered twice daily.

Clause 26. The method of clause 24 or 25, wherein the pharmaceutical dosage form comprises 400 mg ebselen, and oral administration is performed twice daily.

Clause 27. The method of clause 24 or 25, wherein the pharmaceutical dosage form comprises 800 mg ebselen, and oral administration is performed twice daily.

Clause 28. The method of clause 24, wherein each dosage form is a capsule.

Clause 29. The method of clause 25, wherein the dosage form administered as an amorphous form of ebselen exhibits greater mean peak concentration (Cₘₐₓ) after oral administration compared to a dosage form administered as two dry blend capsules each comprising 200 mg ebselen, 120 mg microcrystalline cellulose, 28 mg croscarmellose sodium and 1.8 mg magnesium stearate.

Clause 30. The method of clause 26, wherein the dosage form administered as an amorphous form of ebselen exhibits a mean peak concentration (Cₘₐₓ) at least 3-fold greater Cₘₐₓ than the dosage form administered as compared to two dry blend capsules each comprising 200 mg ebselen, 120 mg microcrystalline cellulose, 28 mg croscarmellose sodium and 1.8 mg magnesium stearate.

### 6. EXAMPLES

The Examples in this section are offered by way of illustration, and not by way of limitation. The examples can represent only some embodiments, and it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

### 6.1. Example 1: Preparation of control ebselen dosage form (200 mg capsule) (“Formulation 1”)

The following control ebselen dosage is a gelatin capsule containing 200 mg of ebselen and 150 mg of excipients microcrystalline cellulose, sodium croscarmellose and magnesium stearate, and has been previously described (see for example, U.S. patent application Ser. No. 16/300,935 (published as US20200261417)).

The quantitative composition of control ebselen dosage from, 200 mg capsules, is described in Table 1.

TABLE 1
Quantitative composition of control
ebselen dosage form (Capsule, 200 mg)

| Material | Function | Capsules (mg) | % w/w |
|---|---|---|---|
| Ebselen | Drug Substance | 200.0 | 47.3 |
| Microcrystalline cellulose | Filler | 120.2 | 28.3 |
| Croscarmellose Sodium | Disintegrant | 28.0 | 6.6 |
| Magnesium Stearate | Lubricant | 1.8 | 0.4 |
| White Opaque Hard Gelatin | Capsule Shell | 73.7 | 17.4 |
| Capsule, Size #1 | | | |
| Target Capsule Weight | | 423.7 | 100.0 |

Components except magnesium stearate are individually weighed, manually screened, and then blended. Magnesium stearate is screened, added to the blender and mixed with the previously blended components. The resulting blend is filled into the capsule shells to the appropriate fill weight.

Blend was discharged into low density polyethylene bags labeled and transferred to the encapsulator for encapsulation in gelatin capsules.

The control ebselen dosage form was obtained as capsules containing 200 mg of ebselen.

### 6.2. Example 2: Ebselen spray-dried dispersion and hot melt extrusion formulation (“ebselen ASD/HME” or “Formulation 2”)

Method: Amorphous solid dispersion (ASD) of ebselen with drug load of 20% (w/w) was developed using methods of rotary evaporation, spray drying (SD) and hot-melt extrusion (HME). Different kinds of polymers were selected as carrier materials and ASDs were prepared via rotary evaporation, spray drying and hot-melt extrusion. Obtained products were characterized with PLM, XRPD and TGA/mDSC, and further evaluated by solubility and stability tests.

The following polymers were selected for amorphous solid dispersion (ASD) development of ebselen by rotary evaporation: polyvinylpyrrolidone (PVP K30, “polymer 1”), copovidone (PVP VA64 or Kollidon VA64, “polymer 2”), (Hydropropyl)methyl cellulose (HPMC E3, “polymer 3”), (Hydropropyl)methyl cellulose (HPMC ASLF, “polymer 4”), polyvinyl caprolactam-polyvinyl acetate-polyethylene glycol graft co-polymer (soluplus, “polymer 5”), Poly(butyl methacrylate-co-(2-demethylaminoeethyl) methacrylate-co-methyl methacrylate) 1:2:1 (Eudragit EPO, “polymer 6”), Poly(methacrylic acid-co-methyl methacrylate) 1:1 (Eudragit L100, “polymer 7”), and Polyethylene glycol (PEG 8000, “polymer 8”). Amorphous solid dispersions of an amorphous form of ebselen having compositions set forth in table 2 were prepared by spray drying:

TABLE 2
ASD compositions made by rotary evaporation

| | A | B | C | D | E | F | G | H |
|---|---|---|---|---|---|---|---|---|
| Ingredients | % w/w | % w/w | % w/w | % w/w | % w/w | % w/w | % w/w | % w/w |
| ebselen | 20 | 20 | 20 | 20 | 20 | 20 | 20 | 20 |
| Polymer 1 | 80 | | | | | | | |
| Polymer 2 | | 80 | | | | | | |
| Polymer 3 | | | 80 | | | | | |
| Polymer 4 | | | | 80 | | | | |
| Polymer 5 | | | | | 80 | | | |
| Polymer 6 | | | | | | 80 | | |
| Polymer 7 | | | | | | | 80 | |
| Polymer 8 | | | | | | | | 80 |

Characterization and evaluation of formulations in Table 2 indicated that ASD products with polymer 6 (methacrylate-co-methyl methacrylate) 1:2:1, Eurdragit EPO) showed significant solubility enhancement.

Based on previous data, three kinds of polymers including (Hydropropyl)methyl cellulose (HPMC ASLF, “polymer 4”), polyvinyl caprolactam-polyvinyl acetate-polyethylene glycol graft co-polymer (soluplus, “polymer 5”), and poly(butyl methacrylate-co-(2-demethylaminoeethyl) methacrylate-co-methyl methacrylate) 1:2:1 (Eudragit EPO, “polymer 6”), were selected as carrier materials for amorphous solid dispersion (ASD) of ebselen prepared via spray drying. THF was selected as desired solvent system owing to its good solubility.

Amorphous solid dispersions of an amorphous form of ebselen having compositions set forth in table 3 were prepared by spray drying:

TABLE 3
ASD compositions made by spray drying

| | I | J | K |
|---|---|---|---|
| Ingredients | % w/w | % w/w | % w/w |
| ebselen | 20 | 20 | 20 |
| Polymer 4 | 80 | | |
| Polymer 5 | | 80 | |
| Polymer 6 | | | 80 |
| THF | q.s. | q.s. | q.s. |

q.s. = quantity sufficient

Amorphous material was obtained for Formulation J (ebselen and polymer 5 (polyvinyl caprolactam-polyvinyl acetate-polyethylene glycol graft co-polymer (soluplus)) after spray-drying. However, crystalline material was obtained for Formulation I (ebselen and polymer 4 ((Hydropropyl)methyl cellulose (HPMC ASLF)), and Formulation K (ebselen and polymer 6 (poly(butyl methacrylate-co-(2-demethylaminoeethyl) methacrylate-co-methyl methacrylate) 1:2:1 (Eudragit EPO)).

Compared with pure ebselen, the spray-dried products exhibited an obvious solubility enhancement, especially spray-dried products of Formulation J, which indicated solubility of above 90 μg/mL in bio-relevant media.

Next, amorphous solid dispersion (ASD) preparation of ebselen was continued via method of hot-melt extrusion. three kinds of polymers including (Hydropropyl)methyl cellulose (HPMC ASLF, “polymer 4”), polyvinyl caprolactam-polyvinyl acetate-polyethylene glycol graft co-polymer (soluplus, “polymer 5”), and copovidone (PVP VA64 or Kollidon VA64, “polymer 2”) were selected as carrier materials for amorphous solid dispersion (ASD) of ebselen prepared via hot-melt extrusion.

Amorphous solid dispersions of an amorphous form of ebselen having compositions set forth in table 4 were prepared by hot-melt extrusion:

TABLE 4
ASD compositions made by hot-melt extrusion

| | L | M | N |
|---|---|---|---|
| Ingredients | % w/w | % w/w | % w/w |
| ebselen | 20 | 20 | 20 |
| Polymer 4 | 79.3 | | |
| Polymer 5 | | 79.3 | |
| Polymer 2 | | | 79.3 |
| methyl cellulose (MC) | 0.5 | 0.5 | 0.5 |
| polysorbate 80 | 0.2 | 0.2 | 0.2 |

Amorphous solid dispersions (ASDs) of an amorphous form of ebselen were prepared successfully, and ASD obtained with polyvinyl caprolactam-polyvinyl acetate-polyethylene glycol graft co-polymer (soluplus, “polymer 5”), and copovidone (PVP VA64 or Kollidon VA64, “polymer 2”) exhibited obvious solubility enhancement. However, ASD obtained with polymer 5 exhibited poor physical stability after 2 weeks, whereas ASD obtained with polymer 2 exhibited good physical and chemical stability up to 2 weeks.

Next, suspension formulation preparation and evaluation of ASD of ebselen (20% w/w) with copovidone (polymer 2) was performed in 0.5% MC/0.2% polysorbate 80 (referred to herein as “Formulation 2”). Good uniformity and chemical stability were observed for Formulation 2.

In summary, suspension formulation of ASD of an amorphous form of ebselen with copovidone (polymer 2) in 0.5% MC/0.2% polysorbate 80 was acceptable for further development and designated “Formulation 2” (also referred to herein as ebselen spray-dried dispersion and hot melt extrusion formulation (ebselen ASD/HME)).

### 6.3. Example 3: Minipig pharmacokinetic study of Formulation 1 (control) vs Formulation 2 (ebselen ASD/HME)

Method: The purpose of this study was to determine the pharmacokinetics of ebselen and its metabolites in male and female bama mini-pigs following twice daily (BID) oral administration of two test formulations, control ebselen capsules (Formulation 1) and ebselen-ASD/HME capsules (Formulation 2) at 200 mg/animal for 2.5 days. Three male and three female naïve Bama mini-pigs were assigned to this study. In phase 1, animals received five oral doses (BID on day 1, BID on day 2 and QD on day 3) of Formulation 1: each dose comprised control ebselen capsules (1 (Ebselen capsule, 200 mg+3 placebo capsules). After one week wash out period, the same animals received five oral doses (BID on day 1, BID on day 2 and QD on day 3) of Formulation 2: each dose comprised four Ebselen ASD/HME capsules (Formulation 2), 50 mg each. In each phase, there were 12 hour intervals between dosing. Blood samples were collected at 0.5, 1, 2, 3, 4, 6, 8 and 12 hours post the first and fifth dosing. Concentrations of ebselen, and ebselen glucuronide metabolite in plasma samples were determined by LC-MS/MS. A pharmacokinetic study of ebselen, following a single oral administration of control ebselen capsules (Formulation 1) or ebselen-ASD/HME capsules (Formulation 2) at 200 mg/animal, ebselen showed a gender-averaged AUC (-12h of 1715±732, 4544±2009 hong/mL, respectively. The Cₘₐₓ values were 410±340, 1225±604 ng/ml and while Tₘₐₓ reached 5.67±0.816, 3.17±1.83 h, respectively. T_{1/2} were 1.99±0.854, 1.91±0.578 h, respectively. Systemic exposure ratios of female to male were 1.18 for AUC₀₋ₗₐₛₜ and 1.96 for Cₘₐₓ after single oral administration of ebselen control capsules (Formulation 1) 5 capsules, and ratios of female to male were 0.721 and 0.725 for AUC₀₋ₗₐₛₜ and Cₘₐₓ after single oral administration of ASD/HME capsules (Formulation 2), indicating ebselen had no marked sex differences in systemic exposure. The systemic exposure (AUC₀₋ₗₐₛₜ and Cₘₐₓ) to ebselen increased 2- to 3-fold after dosing with ASD/HME capsules (Formulation 2) when compared with control ebselen capsules (Formulation 1). After five consecutive doses of control ebselen capsules (Formulation 1) or ebselen-ASD/HME capsules (Formulation 2) over 2.5 days, no notable change was observed on the systemic exposure (AUC₀₋ₗₐₛₜ and Cₘₐₓ) to ebselen in male and female mini-pigs. Following a single oral administration of control ebselen capsules (Formulation 1) or ebselen-ASD/HME capsules (Formulation 2) at 200 mg/animal, glucuronidated ebselen showed a gender-averaged AUC₀₋₁₂ₕ of 5924±1612, 13082±5370 h·ng/mL, respectively. The Cₘₐₓ values were 1113±374, 3922±2462 ng/ml and while Tₘₐₓ reached 4.83±1.83, 2.75±2.04 h, respectively. T_{1/2} were 2.79±1.20, 2.096±0.533 h. The systemic exposure (AUC₀₋ₗₐₛₜ and Cₘₐₓ) to glucuronidated ebselen was 2 to 4-fold higher after dosing with ASD/HME capsules (Formulation 2) when compared with control ebselen capsules (Formulation 1). After five consecutive doses of control ebselen capsules (Formulation 1) or ebselen-ASD/HME capsules (Formulation 2) over 2.5 days, no notable change was observed on the systemic exposure (AUC₀₋ₗₐₛₜ and Cₘₐₓ) to glucuronidated ebselen in male and female mini-pigs after the 5th dose.

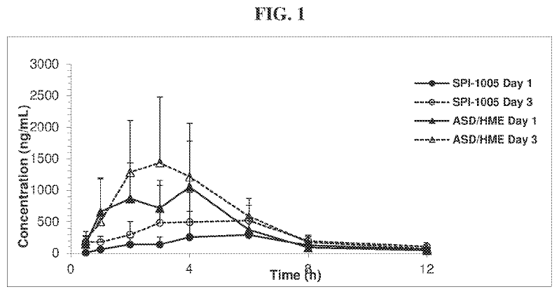

FIG. 1 shows plasma concentrations of ebselen after oral (PO) administration of control Formulation 1 (referred to as SPI-1005) or Formulation 2, ebselen amorphous spray-dried dispersion hot melt extrusion formulation (referred to as ASD/HME).

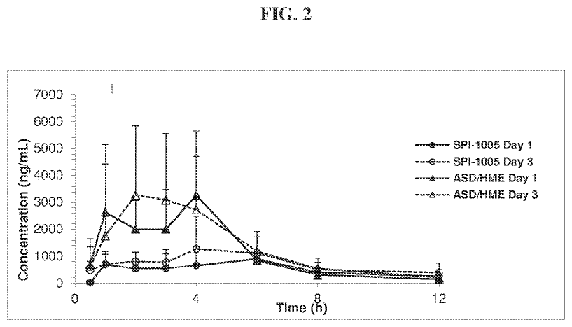

FIG. 2 shows plasma concentrations of ebselen glucuronide after oral (PO) administration of control Formulation 1 (referred to as SPI-1005) or Formulation 2, ebselen amorphous spray-dried dispersion hot melt extrusion formulation (referred to as ASD/HME).

Tables 5 and 6 below also summarize the ebselen levels and glucuronide levels observed respectively in male and female Bama minipigs on days 1 and 3 after oral dosing with Formulations 1 and 2 twice daily on day 1, day 2 and day 3.

TABLE 5
Ebselen levels in male and female Bama minipigs after oral dosing
of two formulations BID day 1, BID day 2 and QD day 3.

| Formulation | Sample | | Cₘₐₓ | Cₘₐₓ | AUC₀₋ₗₐₛₜ | AUC₀₋ₗₐₛₜ |
|---|---|---|---|---|---|---|
| # | Collection Day | Gender | values | Ratio | values | ratio |
| 1 | Day 1 | F/M | 542/277 | 1.96 | 1860/1570 | 1.18 |
| | Day 3 | F/M | 1011/521 | 1.94 | 3645/3167 | 1.15 |
| 2 | Day 1 | F/M | 1029/1420 | 0.725 | 3807/5281 | 0.721 |
| | Day 3 | F/M | 1917/1547 | 1.24 | 7317/5748 | 1.27 |

TABLE 6
Ebselen glucuronide levels in male and female Bama minipigs after
oral dosing of two formulations BID day 1, BID day 2 and QD day 3.

| Formulation | Sample | | Cₘₐₓ | Cₘₐₓ | AUC₀₋ₗₐₛₜ | AUC₀₋ₗₐₛₜ |
|---|---|---|---|---|---|---|
| # | Collection Day | Gender | values | Ratio | values | ratio |
| 1 | Day 1 | F/M | 542/277 | 1.96 | 1860/1570 | 1.18 |
| | Day 3 | F/M | 1011/521 | 1.94 | 3645/3167 | 1.15 |
| 2 | Day 1 | F/M | 1029/1420 | 0.725 | 3807/5281 | 0.721 |

In summary, the systemic exposures (AUC₀₋ₗₐₛₜ and Cₘₐₓ) to ebselen and ebselen glucuronide were greater after dosing with ebselen-ASD/HME capsules (Formulation 2) compared to dosing with control ebselen capsules (Formulation 1). No marked gender differences in systemic exposure were observed for ebselen and ebselen glucuronide. No obvious accumulation was observed on systemic exposure to ebselen and ebselen glucuronide in male and female mini-pigs after the 5th dose.

### 7. Equivalents and Incorporation by Reference

While the disclosure has been particularly shown and described with reference to a preferred embodiment and various alternate embodiments, it will be understood by persons skilled in the relevant art that various changes in form and details can be made therein without departing from the spirit and scope of the disclosure.

All publications, patents, patent applications, and other documents cited in this application, including U.S. Provisional Appl. No. 63/349,474, are hereby incorporated by reference in their entireties for all purposes to the same extent as if each individual publication, patent, patent application, or other document were individually indicated to be incorporated by reference for all purposes.

What is claimed is:

1. An amorphous solid dispersion (ASD) comprising an amorphous form of ebselen and a carrier polymer.

2. The amorphous solid dispersion of claim 1, comprising 10-30% w/w of ebselen.

3. The amorphous solid dispersion of claim 2, comprising 15-25% w/w of ebselen.

4. The amorphous solid dispersion of claim 3, comprising about 20% w/w ebselen.

5. The amorphous solid dispersion of any one of claims 1 to 4, wherein the carrier polymer is selected from a polyvinylpyrrolidone polymer, a copovidone polymer, a hydroxypropyl methyl cellulose polymer, a polyvinyl caprolactam-polyvinyl acetate-polyethylene glycol graft co-polymer, a dimethylaminoethyl methacrylate-copolymer, a methacrylic acid-methyl methacrylate copolymer, and a polyethylene glycol polymer.

6. The amorphous solid dispersion of claim 5, wherein the carrier polymer is a copovidone polymer, a hydroxypropyl methyl cellulose polymer, a polyvinyl caprolactam-polyvinyl acetate-polyethylene glycol graft co-polymer, or a combination thereof.

7. The amorphous solid dispersion of claim 6, wherein the carrier polymer is a copovidone polymer.

8. The amorphous solid dispersion of any one of claims 1 to 7, wherein the solid dispersion is formed by hot melt extrusion of a mixture comprising ebselen and a carrier protein.

9. A pharmaceutical composition comprising the amorphous solid dispersion of any one of claims 1 to 8 and one or more pharmaceutically acceptable excipients.

10. The pharmaceutical composition of claim 9, wherein the one or more excipients comprises a thickener, a solubilizer, a surfactant an emulsifier, or a combination thereof.

11. The pharmaceutical composition of claim 9 or 10, further comprising methyl cellulose.

12. The pharmaceutical composition of any one of claims 9 to 11, further comprising polyethylene sorbitol ester.

13. The pharmaceutical composition of any one of claims 9 to 12, further comprising a mixture of methyl cellulose and polyethylene sorbitol ester.

14. A pharmaceutical dosage form comprising the amorphous solid dispersion of any one of claims 1 to 8, or a pharmaceutical composition of any one of claims 9 to 13.

15. The pharmaceutical dosage form of claim 14, in the form of a powder, rod, tablet, capsule, or any other form that results from spray drying, hot melt extrusion or high shear mixing of the amorphous solid dispersion.

16. The pharmaceutical dosage form of claim 14 or 15, wherein the ebselen is present in an amount of from 50 mg to 200 mg.

17. The pharmaceutical dosage form of claim 16, wherein the ebselen is present in an amount of about 50 mg.

18. The pharmaceutical dosage form of claim 16, wherein the ebselen is present in an amount of about 100 mg.

19. The pharmaceutical dosage form of claim 16, wherein the ebselen is present in an amount of about 200 mg.

20. The pharmaceutical dosage form of claim 16, wherein the ebselen is present in an amount of about 400 mg.

21. The pharmaceutical dosage form of claim 14, wherein the dosage form comprises 50 mg ebselen amorphous spray-dried dispersion in hot melt extrusion of copovidone 79.3%, 0.5% methyl cellulose and 0.2% polyethylene sorbitol ester.

22. The pharmaceutical dosage form of claim 21, wherein the dosage form is a capsule.

23. The pharmaceutical dosage form of any one of claims 14 to 22, wherein oral administration of said pharmaceutical dosage form to a selected human subject group produces in said selected human subject group:
an enhanced maximum blood plasma concentration (Cₘₐₓ) for ebselen that is at least 2-fold greater than that achieved with a control ebselen formulation;
an area under the curve (AUC) for ebselen that is at least 2-fold greater than that achieved with a control ebselen formulation; and
an area under the curve (AUC) for ebselen metabolite that is at least 2-fold greater than that achieved with a control ebselen formulation.

24. A method of delivering a therapeutically effective amount of ebselen to a subject in need thereof, comprising orally administering to a subject in need thereof a pharmaceutical dosage form according to any one of claims 14 to 23, to achieve:
an enhanced maximum blood plasma concentration (Cₘₐₓ) for ebselen that is at least 2-fold greater than that achieved with a control ebselen formulation;
an area under the curve (AUC) for ebselen that is at least 2-fold greater than that achieved with a control ebselen formulation
an area under the curve (AUC) for ebselen metabolite that is at least 2-fold greater than that achieved with a control ebselen formulation.

25. The method of claim 24, wherein the pharmaceutical dosage form is administered twice daily.

26. The method of claim 24 or 25, wherein the pharmaceutical dosage form comprises 400 mg ebselen, and oral administration is performed twice daily.

27. The method of claim 24 or 25, wherein the pharmaceutical dosage form comprises 800 mg ebselen, and oral administration is performed twice daily.

28. The method of claim 24, wherein each dosage form is a capsule.

29. The method of claim 25, wherein the dosage form administered as an amorphous form of ebselen exhibits greater mean peak concentration (Cₘₐₓ) after oral administration compared to a dosage form administered as two dry blend capsules each comprising 200 mg ebselen, 120 mg microcrystalline cellulose, 28 mg croscarmellose sodium and 1.8 mg magnesium stearate.

30. The method of claim 26, wherein the dosage form administered as an amorphous form of ebselen exhibits a mean peak concentration (Cₘₐₓ) at least 3-fold greater Cₘₐₓ than the dosage form administered as compared to two dry blend capsules each comprising 200 mg ebselen, 120 mg microcrystalline cellulose, 28 mg croscarmellose sodium and 1.8 mg magnesium stearate.
